(19) United States
(12) Patent Application Publication
(10) Pub. No.: US 2024/0415802 A1
(43) Pub. Date: Dec. 19, 2024

(54) GENISTEIN FOR ASTHMA TREATMENT

(71) Applicants: University of South Florida, Tampa, FL (US); Humanetics Corporation, Minneapolis, MN (US); Ann & Robert H. Lurie Children's Hospital of Chicago, Chicago, IL (US)

(72) Inventors: Seong H. Cho, Tampa, FL (US); Rajesh Kumar, Chicago, IL (US); Brett D. Anderson, Minneapolis, MN (US); Michael D. Kaytor, Maplewood, MN (US)

(21) Appl. No.: 18/814,657

(22) Filed: Aug. 26, 2024

Related U.S. Application Data
(63) Continuation of application No. PCT/US2023/015204, filed on Mar. 14, 2024.
(60) Provisional application No. 63/269,275, filed on Mar. 14, 2022.

(51) Int. Cl. A61K 31/352; A61P 11/06; C12Q 1/6883

(52) U.S. Cl. CPC A61K 31/352; A61P 11/06; C12Q 1/6883; C12Q 2600/106; C12Q 2600/156

(57) ABSTRACT

A novel method of treating diseases characterized by high PAI-1 is presented. Administering a composition comprising a therapeutically effective amount of genistein was found to inhibit PAI-1 promotor activity and decrease PAI-1 levels to treat diseases such as asthma, which exhibit increased PAI-1 levels. Genotyping the patient can be performed prior to administration to detect a 4G/5G polymorphism as patients having a 4G genotype have better response to the genistein treatment. The composition may be comprised of a therapeutically effective amount of genistein derived naturally from soybeans or synthetically produced and optionally present in nanoparticle form.

### CROSS REFERENCE TO RELATED APPLICATIONS

This application is a continuation of and claims priority to International Application Serial No. PCT/US2023/015204, entitled “Genistein for Asthma Treatment” filed Mar. 14, 2023, which claims priority to U.S. Provisional Patent Application Ser. No. 63/269,275, entitled “Genistein for Asthma Prevention and Treatment”, filed Mar. 14, 2022, the contents of which are hereby incorporated by reference into this disclosure.

### FIELD OF INVENTION

This invention relates to treatment of diseases associated with high PAI-1. More specifically, the present invention provides therapeutic methods and compositions for treating high PAI-1 diseases such as asthma.

### BACKGROUND OF THE INVENTION

Plasminogen activator inhibitor-1 (PAI-1) has been reported to be involved in the pathogenesis of various diseases.^{1,2} The inventors previously reported that PAI-1 plays an important role in the pathogenesis of asthma, that Mast Cells (MCs) are a major source of PAI-1 in asthma and that PAI-1 levels are increased in the airways in mouse models of asthma.^{3,4,5} The previous study also showed that PAI-1 inhibition with Tiplaxtinin reduced the degree of cosinophilic airway inflammation and Airway hyperresponsiveness (AHR) in an Ovalbumin (OVA) challenge model of allergic asthma.⁶ Miyamoto et al. reported that intra-airway administration of a small interfering RNA against PAI-1 suppressed cosinophilic airway inflammation and AHR in a mouse model of acute asthma.⁷ The downregulation of TGF-β1-induced PAI-1 production from normal bronchial epithelial cells by genistein treatment suggests that genistein may reduce PAI-1 production in the airways of asthmatics.⁸ Therefore genistein, a major soy isoflavone, was thought to be a promising therapeutic candidate in the treatment of asthma. However, a recent multicenter, placebo-controlled, randomized clinical trial study examining soy isoflavone supplements compared with placebo did not improve lung function or clinical outcomes in the entire population.⁹

One of the reasons for the negative results in the soy isoflavone trial may be intrinsic individual differences in medication responses because of genetic variation. As it is well known that there are various endotypes of asthma,¹⁰ PAI-1 may play an important role only in the pathogenesis of asthmatics with specific genotypes. Plasma PAI-1 levels have been found to be higher in patients with the 4G4G genotype than in those with the 5G5G genotype, with the 4G5G group having intermediate values.¹¹ The inventors recently reported that soy isoflavone administration significantly reduces the number of severe asthma exacerbations only in asthmatic patients with the 4G-containing genotype.⁸ These results suggest that PAI-1 plays an important role mainly in asthmatics with the high PAI-1 producing genotype. Still, the detailed mechanism of enhanced PAI-1 production in subjects with the 4G allele and how genistein reduces PAI-1 production remains to be elucidated.

### SUMMARY OF THE INVENTION

The 4G/5G polymorphism of plasminogen activator inhibitor 1 (PAI-1) is associated with elevated plasma PAI-1 levels and poor asthma control. Genistein, a major soy isoflavone, reduces severe asthma exacerbations in patients with the 4G allele by reducing PAI-1. Genistein reduces allergic inflammation and improves lung function when used as treatment for asthma. The inventors found a difference in PAI-1 production from asthmatics with 4G and 5G allele can result from a differential transcriptional regulation of the PAI-1 polymorphism and that genistein can reduce upregulated PAI-1 production via blocking the transcriptional activation of the 4G promoter. The inventors found that genistein treatment, whether natural or synthetic genistein is used, is efficacious when treating patients having 4G4G or 4G5G genotype. The inventors found that synthetic genistein in a pharmaceutical composition such as Bio 300™, was efficacious in vivo in an asthma model of mice to decrease PAI-1 levels.

In an embodiment, a method of decreasing plasminogen activator inhibitor 1 (PAI-1) levels in a patient having a disease characterized by increased plasminogen activator inhibitor 1 (PAI-1) as compared to a normal control is presented comprising administering a therapeutically effective amount of a composition comprising nanoparticle genistein and at least one pharmaceutically acceptable carrier to the patient. The disease may be asthma.

The method may also be comprised of performing or having performed genotype analysis of the PAI-1 gene in the patient prior to administration of the composition. In some embodiments, the composition is only administered to the patient if the patient is determined to have a 4G allele of the PAI-1 gene.

In some embodiments, the composition is BIO 300™ and the therapeutically effective amount is between about 500 mg to about 1500 mg.

In another embodiment, a method of inhibiting PAI-1 promotor activity in a patient having a disease characterized by increased plasminogen activator inhibitor 1 (PAI-1) as compared to a normal control is presented comprising: performing or having performed genotype analysis of the plasminogen activator inhibitor 1 (PAI-1) gene; determining or having determined presence of a 4G/5G polymorphism in the PAI-1 gene promoter region; and administering a therapeutically effective amount of a composition comprising genistein to the patient if the 4G/5G polymorphism is present. The composition may be further comprised of at least one pharmaceutically acceptable carrier with the therapeutically effective amount of the genistein is at least 32 mg. The disease may be asthma, atopic dermatitis, chronic sinusitis, eosinophilic esophagitis, idiopathic pulmonary fibrosis, lung injury associated fibrosis, severe acute respiratory syndrome coronavirus 2 (SARS-COV-2 or COVID-19), atherosclerosis, thrombosis, metabolic syndrome, lung cancer, obesity, type 2 diabetes, hypertension, or cardiovascular disease.

In a further embodiment, a method of treating a disease characterized by increased plasminogen activator inhibitor 1 (PAI-1) levels in a patient in need thereof is presented comprising: performing or having performed genotype analysis of the plasminogen activator inhibitor 1 (PAI-1) gene; and administering a therapeutically effective amount of a composition comprising genistein to the patient if the patient has a 4G allele.

The composition may further comprise at least one pharmaceutically acceptable carrier with the therapeutically effective amount of genistein being at least 32 mg.

Alternatively, the composition is comprised of nanoparticle genistein and at least one pharmaceutically acceptable carrier. In this embodiment, the composition may be BIO 300™ and the therapeutically effective amount may be between about 500 mg to about 1500 mg.

The disease may be asthma, atopic dermatitis, chronic sinusitis, cosinophilic esophagitis, idiopathic pulmonary fibrosis, lung injury associated fibrosis, severe acute respiratory syndrome coronavirus 2 (SARS-COV-2 or COVID-19), atherosclerosis, thrombosis, metabolic syndrome, lung cancer, obesity, type 2 diabetes, hypertension, or cardiovascular disease.

### BRIEF DESCRIPTION OF THE DRAWINGS

For a fuller understanding of the invention, reference should be made to the following detailed description, taken in connection with the accompanying drawings, in which:

FIG. 1A-B are a series of graphs depicting genistein inhibits PAI-1 gene polymorphism (4G) promoter activity in Normal Human Bronchial Epithelial Cells (NHBE). (A) NHBE cells were cultured and transfected with luciferase plasmids containing different PAI-1 promoters, polymorphisms, 4G, 5G and pGL3-basic vector (empty backbone). Cells were stimulated for 6 hours with TGF-β (A) with and without genistein pretreatment (5 μM) for 1 hour. (B) PAI-1 4G promoter activity was significantly induced by TGF-β stimulation compared to no treatment (p=0.001) (A). PAI-1 4G promoter activity was significantly reduced by genistein pretreatment (p=0.007). The data in the graph is as means±SE, ** p<0.01.

FIG. 2A-B are a series of graphs depicting genistein inhibits PAI-1 gene polymorphism (4G) promoter activity in Human mast cells (LAD2). (A) LAD2 cells were cultured and transfected with luciferase plasmids containing different PAI-1 promoter polymorphisms, 4G, 5G and pGL3-basic vector (empty backbone). Cells were stimulated for 30 min with IgE+Streptavidin/Ionomycin+PMA (A) with and without genistein pretreatment (5 μM) for 1 hour (B). PAI-1 4G promoter activity was significantly induced by IgE+Streptavidin/Ionomycin+PMA stimulation. Transfection with PAI-1 5G and basic vector in LAD2 cells didn't show increase of promoter activity by stimulation. (B) These increased promoter activities by IgE+Streptavidin/Ionomycin+PMA stimulation in LAD2 cells transfected with 4G vector were blocked by genistein pretreatment significantly. The data in the graph is expressed as means±SE, *p<0.05 and ** p<0.005.

FIG. 2C-D are a series of graphs depicting transfection with PAI-1 5G and basic vector in LAD2 cells didn't show increase of promoter activity by stimulation.

FIG. 3 is an image depicting the experimental plan for the nanoparticulate genistein in vivo studies.

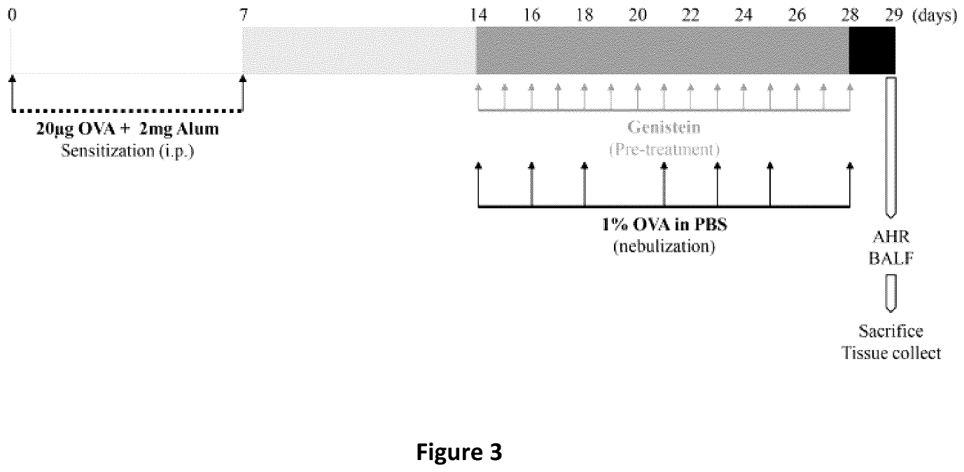

FIG. 4 is a series of graphs depicting cell counting in BALF. Lymphocytes, Eosinophils and Neutrophils positive cells were significantly decreased in OVA+BIO 300™ BIO 300™ versus OVA+Veh (**** p<0.0001). Eosinophils/Lymphocyte results showed a decreased ratio in OVA+BIO 300™ BIO 300™ as compared to OVA+Veh (*p=0.0253). Total cell count result showed a significant decrease in OVA+BIO 300™ BIO 300™ vs OVA+Veh (*** p=0.0006).

FIG. 5A is a series of images depicting cell counts in tissue samples.

FIG. 5B is a series of graphs depicting Eosinophils and Eosinophils/Lymphocyte result showed a significant decrease in OVA+BIO 300™ BIO 300™ versus OVA+Veh (**** p<0.0001). Lymphocytes and Neutrophils positive cells also exhibited a decreased pattern in OVA+BIO 300™ BIO 300™ vs OVA+Veh. Total cell count result showed significantly decreased OVA+BIO 300™ BIO 300™ vs OVA+Veh (*** p=0.0006). Scale bar=50 um

FIG. 6 is a series of images depicting PAS stain of lung tissue in each group. Compared with the control group, OVA+Veh groups showed increased goblet cell (**** p<0.0001). Furthermore, goblet cell number was significantly decreased in OVA+BIO 300™ BIO 300™ versus OVA+Veh (**** p<0.0001). Scale bar=50 um.

FIG. 7 is a series of images depicting Trichrome stain of lung tissue in each group. Compared with the control group, OVA+Veh groups showed increased goblet cell (**** p<0.0001). Furthermore, goblet cell number was significantly decreased in OVA+BIO 300™ BIO 300™ versus OVA+Veh (**** p<0.0001). Scale bar=50 um.

FIG. 8 is a series of graphs depicting AHR result for Rrs average (top) and following methacholine dose, Rrs measurement (bottom). In 25 mg/ml methacholine, Rrs percentage was significantly decreased in OVA+BIO 300™ BIO 300™ versus OVA+Veh (*p=0.0433). In 50 mg/ml dose, percentage was double time decreased OVA+BIO 300™ BIO 300™ versus OVA+Veh (*** p=0.002).

FIG. 9 is a graph depicting BALF PAI-1 result. The effect of BIO 300™ BIO 300™ treatment of plasminogen activator inhibitor (PAI-1) in BAL fluid (BALF) samples from phosphate-buffered saline (PBS) or OVA-challenged mice. OVA+BIO 300™ BIO 300™ mice were treated with BIO 300™ BIO 300™ during OVA challenge and PAI-1 assessed by ELISA. Data are shown as mean±SEM (n=7 mice/group). * P=0.0136 between groups with Mann-Whitney test (GraphPad Prism 9.0 program).

FIG. 10 is a graph depicting the level of PAI-1 in mice lung homogenates by ELISA assay. PAI-1 levels in lung homogenate supernatants were determined by ELISA. PAI-1 concentration was significantly decreased in BIO 300™ BIO 300™ treated OVA mice compared to OVA+Veh group. The data are presented as means±SE; n=9 mice in PBS+Veh group, n=10 mice in PBS+BIO 300™ BIO 300™ group, n=8 mice in OVA+Veh and OVA+BIO 300™ BIO 300™ groups, *** p=0.0002, ** p=0.007 (Mann-Whitney test).

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention. The following description is not intended to limit the scope of the present description disclosed herein.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supersedes any disclosure of an incorporated publication to the extent there is a contradiction.

All numerical designations, such as pH, temperature, time, concentration, and molecular weight, including ranges, are approximations which are varied up or down by increments of 1.0, 0.1, 0.01, or 0.001 as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, “about” means approximately or nearly and in the context of a numerical value or range set forth means±15% of the numerical.

“Optional” or “optionally” means that the subsequently described event or circumstance may or may not occur, and that the description includes instances where said event or circumstance occurs and instances where it does not.

As used herein, the singular forms “a,” “an” and “the” include plural referents unless the context clearly dictates otherwise. Thus, for example, reference to “a polypeptide” includes a mixture of two or more polypeptides and the like.

As used in this specification and the appended claims, the term “or” is generally employed in its sense including “and/or” unless the context clearly dictates otherwise.

As used herein “patient” is used to describe an animal, preferably a human, to whom treatment is administered, including prophylactic treatment with the compositions of the present invention. The terms “patient” and “subject” are used interchangeably herein.

As used herein “animal” means a multicellular, eukaryotic organism classified in the kingdom Animalia or Metazoa. The term includes, but is not limited to, mammals. Non-limiting examples include humans, rodents, mammals, aquatic mammals, domestic animals such as dogs and cats, farm animals such as sheep, pigs, cows and horses. Wherein the terms “animal” or the plural “animals” are used, it is contemplated that it also applies to any animals.

The terms “risk or susceptibility” as used herein refers to the determination as to whether a subject would or would not respond to a particular therapy or would or would not develop a particular disease or symptom.

The term “normal” as used herein refers to a sample or patient which is assessed as not having high PAI-1, asthma, or any other lung disorder.

“Sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumor lysates, and tissue culture medium, tissue extracts such as homogenized tissue, tumor tissue, cellular extracts, and combinations thereof.

The term “cell” or “cells” is used synonymously herein and refers to in vitro cultures of mammalian cells grown and maintained as known in the art, as well as biological samples obtained from disease specimens or normal specimens in vivo.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue. In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

The term “expression profile” as used herein refers to a genomic expression profile, for example an expression profile of microRNAs or proteins. The profile may be generated by any means for determining a level of a nucleic acid sequence, e.g. quantitative hybridization of microRNA, labeled microRNA, amplified microRNA, cDNA, quantitative PCR, ELISA for quantitation, etc. For proteins, the profiles may be generated by any means for determining a level of a protein, e.g. Western blot, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF), mass spectrometry, microcytometry, microarray, microscopy, fluorescence activated cell sorting (FACS), flow cytometry, and assays based on a property of the protein including but not limited to DNA binding, ligand binding, or interaction with other protein partners. The profile must allow for the analysis of differential gene expression between two samples.

The terms “overexpression” and “underexpression” as used herein refers to the expression of a gene of a patient at a greater or lesser level, respectively, than the normal or control expression of the gene, as measured by gene expression product expression such as mRNA or protein expression, in a sample that is greater than the standard of error of the assay used to assess the expression. A “significant” expression level may be a level which either meets or is above or below a predetermined score for a gene. In some embodiments, expression of the PAI-1 protein is measured and compared to a control sample to determine overexpression and/or 4G/5G polymorphism before treatment with the compositions described herein.

“Administration” or “administering” is used to describe the process in which compounds of the present invention, alone or in combination with other compounds, are delivered to a patient. The composition may be administered in various ways including, but not limited to, orally, nasally, parenterally (referring to intravenous and intraarterial and other appropriate parenteral routes), subcutaneously, etc.

“Parenteral administration” as used herein refers to modes of administration other than enteral and topical administration, usually by injection, and includes, but is not limited to, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion.

“Treatment” or “treating” as used herein refers to any of: the alleviation, amelioration, elimination and/or stabilization of a symptom, as well as delay in progression of a symptom of a particular disorder. For example, “treatment” of a disease associated with high PAI-1, such as asthma, may include any one or more of the following: amelioration and/or elimination of one or more symptoms associated with asthma, reduction of one or more symptoms of asthma, stabilization of symptoms of asthma, and delay in progression of one or more symptoms of asthma.

“Prevention” or “preventing” as used herein refers to any of: halting the effects of asthma, reducing the effects of asthma, reducing the incidence of asthma, reducing the development of asthma, delaying the onset of symptoms of asthma, increasing the time to onset of symptoms of asthma, and reducing the risk of development of asthma.

Exemplary diseases associated with high PAI-1 include, but are not limited to, asthma, atopic dermatitis, chronic sinusitis, eosinophilic esophagitis, idiopathic pulmonary fibrosis, lung injury associated fibrosis, severe acute respiratory syndrome coronavirus 2 (SARS-COV-2 or COVID-19), atherosclerosis, thrombosis, metabolic syndrome, lung cancer, obesity, type 2 diabetes, hypertension, and cardiovascular disease.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, buffers, adjuvants, non-ionic surfactants, preservatives, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W Easton Pennsylvania, Mack Publishing Company, 19^{th} ed.) describes formulations which can be used in connection with the subject invention.

For ease of administration, the subject compounds may be formulated into various pharmaceutical forms. These pharmaceutical compositions are desirably in unitary dosage form suitable, preferably, for administration nasally, orally, rectally, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their case in administration, tablets and capsules often represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution.

A “therapeutically effective amount” as used herein is defined as concentrations or amounts of components which are sufficient to effect beneficial or desired clinical results, including, but not limited to, any one or more of treating symptoms of a disease or disorder exhibiting high PAI-1, including, but not limited to, respiratory diseases such as asthma. While asthma is used as an exemplary disease that can be treated by the compositions herein, any disease or disorder that exhibits high PAI-1 is contemplated for treatment. Compositions of the present invention can be used to effect a favorable change in the condition whether that change is an improvement, such as stopping, reversing, or reducing asthma, or a complete elimination of symptoms or prevention of symptoms due to asthma. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the animal and the route of administration. In some embodiments, the effective amount may vary according to genotype. All ranges given herein include all intervening units to the hundredth percent. The effective range, without regard to genotype, of soy isoflavones may be between about 100-4500 mg/day, or for weight based dosing which may be relevant in certain cases including children, about 1-100 mg/kg/day. In other embodiments, the amount of soy isoflavones may be between about 30-90 mg/kg/day. For those with the PAI-1 variant, the effective range may be from about 0.4 mg/kg to about 40 mg/kg of at least one soy isoflavone per day. In some embodiments, the amount of soy isoflavone per day is between about 0.4-6.2 mg/kg/day. In embodiments where genistein is used in patients with the 4G allele, the effective amount may range from about 0.2 mg/kg/day to 3.1 mg/kg/day in aglycone equivalents. In some embodiments of genistein for treatment regardless of genotype, the treatment range for dosing may be between about 10-100 mg/kg for genistein, or about 500-1500 mg/day. In further embodiments, the amount of genistein used may be at least 32 mg/day with an upper limit of 1500 mg/day. Similarly, if nanoparticulate genistein, such as BIO 300™ is used for treatment regardless of genotype, the treatment range may be between about 10-100 mg/kg/day, or about 500-1500 mg/day. The dose may be adjusted according to response.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. It is contemplated that one of ordinary skill in the art can determine and administer the appropriate dosage of compounds disclosed in the current invention according to the foregoing considerations.

Dosing frequency for the composition includes, but is not limited to, at least about once every three weeks, once every two weeks, once a week, twice a week, three times a week, four times a week, five times a week, six times a week, daily, or twice daily. In some embodiments, the interval between each administration is less than about a week, such as less than about any of 6, 5, 4, 3, 2, 1 or ½ day. In some embodiments, the interval between each administration is constant. For example, the administration can be carried out twice daily, daily, every two days, every three days, every four days, every five days, or weekly. In some embodiments, the administration can be carried out twice daily, three times daily, or more frequently. Administration can also be continuous and adjusted to maintaining a level of the compound within any desired and specified range.

The administration of the composition can be extended over an extended period of time, such as from about a month or shorter up to about three years or longer. For example, the dosing regimen can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The compositions used in the present invention may be administered individually, or in combination with or concurrently with one or more other therapeutics for asthma or other PAI-1 associated diseases. Additionally, compounds used in the present invention may be administered in combination with or concurrently with other therapeutics or preventatives for asthma, other respiratory viruses, or other PAI-1 associated diseases.

“Composition” as used herein refers to a product comprising the specified ingredients in the specified amounts, as well as any product which results, directly or indirectly, from combination of the specified ingredients in the specified amounts. In some embodiments, the composition includes both the therapeutic agent as well as one or more pharmaceutically acceptable carriers. In some embodiments, the composition includes a soy isoflavone, such as genistein, as the therapeutic agent. In some embodiments, the therapeutic agent is a nanoparticulate genistein (NPG) in suspension in at least one pharmaceutically acceptable carrier, e.g. BIO 300™.

“Soy isoflavones” as used herein refers to heterocyclic phenols having structural similarity to estradiol-17ß and selective estrogen receptor modulators. Soy isoflavones can be derived from soybeans. Alternatively, the compounds can be synthetically produced. Exemplary soy isoflavones useful in the instant invention include, but are not limited to, genistein, daidzein, and glycitein.

In some embodiments in which genistein is used, the natural compound sourced from soybeans may be used. Alternatively, a synthetic, purified form of genistein may be used. In some embodiments a nanoparticulate form of genistein is used. Genistein is also referred to under the IUPAC name 5,7-dihydroxy-3-(4-hydroxyphenyl)-chromen-4-onc.

“Nanoparticulate genistein” or “nanoparticle genistein” (NPG) compositions as used herein refers to compositions containing genistein that is in nanoparticulate form, such as BIO 300™ (Humanetics Corporation, Minneapolis, MN). Exemplary NPG compositions include, but is not limited to, those compositions described in U.S. Pat. Nos. 8,900,635, 8,951,560, and 8,551,530. In some embodiments, the nanoparticulate genistein composition used is BIO 300 (Humanetics Corporation, Minneapolis, MN), however other compositions comprising genistein in nanoparticle form are contemplated for use herein.

The 4G4G genotype of plasminogen activator inhibitor 1 (PAI-1) is associated with increased plasma PAI-1 levels and poor asthma control. Previous studies suggest that soy isoflavones can reduce PAI-1 levels. A PAI-1 functional polymorphism (rs1799768, 4G5G) was characterized in subjects with poorly controlled asthma enrolled in a randomized clinical trial of soy isoflavones (n=265). (see PCT/U.S. Pat. No. 2,211,998, herein incorporated by reference in its entirety into this disclosure). Genotype-specific treatment responses on asthma outcomes were compared between soy isoflavones and placebo and the 4G4G/4G5G genotype was found to be associated with a greater risk for allergy-related worsened asthma symptoms and eczema at baseline compared with the 5G5G genotype. There were significant decreases in plasma PAI-1 levels by soy isoflavone treatment, suggesting a biologic connection between genistein intake and plasma PAI-1 levels. Soy isoflavone treatment provides a significant benefit in reducing the number of severe asthma exacerbations in asthmatic patients with the high PAI-1-producing genotype. PAI-1 polymorphisms can be used as a genetic biomarker for soy isoflavone-responsive patients with asthma.

The following non-limiting examples illustrate exemplary compositions and methods of treatment thereof in accordance with various embodiments of the disclosure. The examples are merely illustrative and are not intended to limit the disclosure in any way.

### Example 1-Genistein as a Treatment in High PAI-1 Diseases

As reported previously, mast cells (MCs) are a major source of PAI-1, and MCs produce PAI-1 in response to IgE-dependent activation.⁴ Also, bronchial epithelial cells (ECs) are a major source of PAI-1, and MCs may play an important role in airway remodeling in asthma both as a direct source of PAI-1 and by activating bronchial epithelial cells to produce further PAI-1 via a TGF-β1 mediated activation pathway.⁴

In light of the foregoing, the inventors investigated to confirm that the effect of 4G/5G polymorphism on differential PAI-1 production from these cells is due to differences in PAI-1 gene transcription when MCs or bronchial epithelial cells were stimulated and their promoter activity measured using a luciferase reporter assay.

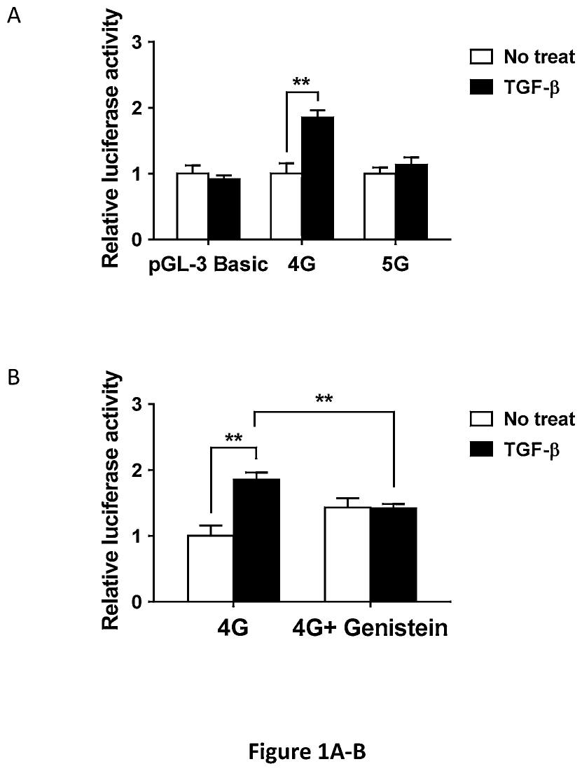

Normal human bronchial epithelial cells (NHBE) and a human mast cell line, LAD2, were cultured and transfected with luciferase plasmids containing a 4G, 5G or pGL3-basic vector (empty backbone). While NHBE cells transfected with the 5G vector didn't increase promoter activity by TGF-β1 stimulation compared to no treatment, NHBE cells transfected with the 4G vector significantly increased promoter activity by TGF-β stimulation compared to no treatment (1.87±0.2-fold, p=0.001). (FIG. 1A) These increased promoter activities by TGF-β1 stimulation in NHBE cells transfected with 4G vector were blocked by genistein pretreatment significantly (p=0.007). (FIG. 1B)

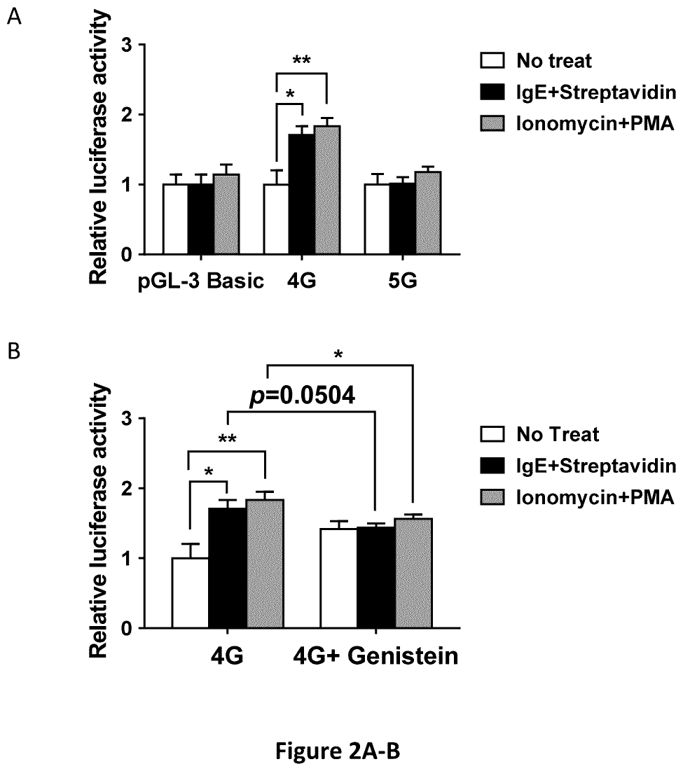

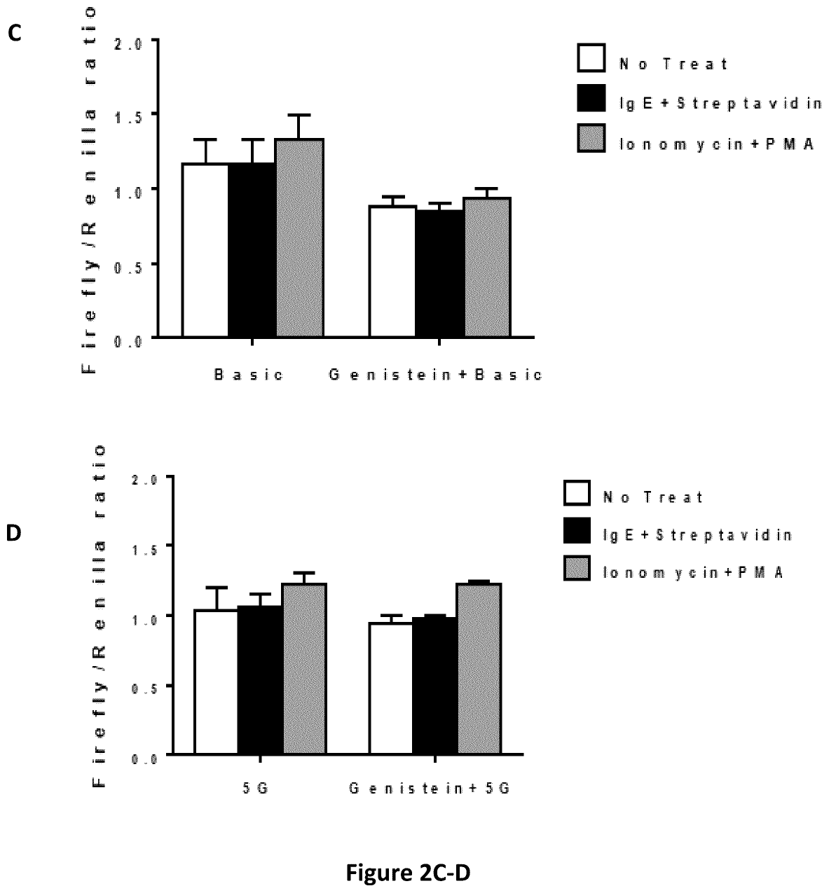

LAD2 cells, a human mast cell line, were cultured and transfected with luciferase plasmids containing a 4G, 5G or pGL3-basic vector to determine the promotor activity. These LAD2 cells were stimulated overnight with IgE (100 ng/mL) and streptavidin (500 ng/mL) (IgE+streptavidin) for IgE-receptor cross-linking or Ionomycin (100 ng/mL) and phorbol myristate acetate (PMA, 10 ng/ml) (Ionomycin+PMA) with or without genistein pretreatment (5 μM) for 1 hour. LAD2 cells transfected with the 4G vector significantly increased promoter activity by IgE+Streptavidin/Ionomycin+PMA stimulation compared to no treatment (1.47±0.2-fold, p=0.001, 1.56±0.2-fold, p=0.006 respectively). (FIG. 2A) These increased promoter activities by IgE+Streptavidin or Ionomycin+PMA stimulation in NHBE cells transfected with 4G vector were blocked by genistein pretreatment significantly (p=0.05 or p<0.05, respectively) (FIG. 2B). LAD2 cells transfected with the 5G vector didn't increase promoter activity by IgE+Streptavidin/Ionomycin+PMA stimulation compared to no treatment (FIGS. 2C and 2D).

The findings highlight a precise mechanism for how genistein reduces PAI-1 production from MCs and bronchial epithelial cells in the context of asthma and allergic disease with the 4G genotype. The experiments have established that the increases in PAI-1 gene promoter activity induced by TGF-β or IgE+streptavidin/Ionomycin+PMA stimulation to NHBE or LAD2 cells occurs specifically in the 4G allele. Genistein reduces PAI-1 production by inhibiting the activation of the PAI-1 promoter activity. The results are consistent with previous findings that soy isoflavone treatment significantly reduces the number of severe asthma exacerbations in asthmatic patients with the 4G/4G or 4G/5G genotype, but not in the 5G/5G genotype.⁸

Over the past decade, the treatment paradigm for non-small cell lung cancer has been altered dramatically. This is mainly due to availability of genetic biomarkers to select patients for targeted treatments.¹² Also, personalized treatment based on biological markers such as IgE, eosinophils and Fractional Exhaled Nitric Oxide (FENO) is increasingly being used in asthma treatment, but personalized treatment based on genetic tests is not common to date.¹³

4G/5G polymorphism in the PAI-1 gene promoter region can be easily measured from blood samples or buccal swab and is considered a minimally invasive test. In addition, as shown herein, genistein is effective in asthma patients with the 4G allele, is inexpensive and has an established safety profile. Genistein can be used as a personalized treatment option based on the genotype in high PAI-1-related diseases such as asthma.

### Methods

### NHBE, Cell Culture, Transfection, and Promotor Assay

Normal human bronchial epithelial cells (NHBE) were cultured in 5% CO₂ chamber at 37° C. with epithelial cell growth basal media (BEGM, Lonza). Cells were seeded on type 1 collagen-coated plate. Prior to transfection and stimulation, NHBEs were cultured without hydrocortisone (HC) for a day or two, at ˜80% confluency in 12 wells. Passage 2 or 3 was used for the experiment. Cells were transfected with luciferase plasmids containing different Pai-1 polymorphisms, 4G4G, 5G5G, pGL3-luciferase reporter basic vector (empty backbone, Promega, Madison, WI, USA) using Lipofectamine 3000 transfection reagent (Invitrogen, Waltham, MA, USA). Cells were stimulated for 6 hours with TGF-β (2 ng/mL, R&D Systems, Minneapolis, MN, USA) with and without genistein (Sigma-Aldrich, St. Louis, MO, USA) pretreatment (5 μM) for 1 hour. Cells were harvested and lysed in 1×PLB reagent prior to performing Dual-luciferase report assay (DLR assay, Promega, Madison, WI, USA). The firefly luciferase activity and Renilla luciferase activity were measured sequentially.

### LAD2, Cell Culture, Transfection and Promotor Assay

Human mast cells (LAD2) were grown in complete serum-free growth media (Stem pro-34 with the supplement, penicillin/streptomycin, L-glutamine and SCF recombinant, Invitrogen, Waltham, MA, USA). Cells were cultured in cytokine-free media (without SCF recombinant) at density of ˜0.5×106 per mL for a day prior to transfection and stimulation. Cells were transfected with luciferase plasmids containing different Pai-1 polymorphisms, 4G4G, 5G5G, pGL3-luciferase reporter basic vector (empty backbone, Promega, Madison, WI) using Lipofectamine 3000 transfection reagent (Invitrogen, Waltham, MA, USA). Cells were sensitized with biotin-conjugated Human IgE (100 ng/mL, Abbiotec, Escondido, CA, USA) overnight and stimulated with streptavidin (500 ng/mL, Invitrogen, Waltham, MA, USA) for 30 min with and without genistein (Sigma-Aldrich, St. Louis, MO, USA) pretreatment (5 μM) for 1 hour. Ionomycin (100 ng/ml, Sigma-Aldrich) and phorbol myristate acetate (PMA, 10 ng/mL, Sigma-Aldrich, St. Louis, MO, USA) were used as positive control. Cells were harvested and lysed in 1×PLB reagent before performing the Dual-luciferase report assay (DLR assay, Promega, Madison, WI, USA). The firefly luciferase activity and Renilla luciferase activity were measured sequentially.

### Conclusion

The 4G/5G polymorphism is an important regulator of PAI-1 production in TGFβ1-stimulated human epithelial cells and IgE-stimulated human mast cells. Genistein reduces PAI-1 production from these cells. This study suggests genistein as a personalized treatment option based on the genotype in high PAI-1-related diseases such as asthma.

### Example 2-Use of Nanoparticulate Genistein as Treatment for PAI-1 Related Diseases

The inventors conducted in vivo experiments on mice to determine the efficacy of a pharmaceutical composition containing nanoparticulate genistein (BIO 300™).

Eight-week-old BALB/c mice were purchased from The Jackson Lab. and housed in standard laboratory conditions (12/12-h light/dark cycle, 22-24° C., 40-60% humidity). Forty female mice were divided into four groups (namely the OVA+Veh, OVA+Bio300, PBS+Veh, PBS+Bio300). The mice in each group (n=10) were equally divided into two cages (5 mice/cage) to provide appropriate space for the mice. Day 0 and Day 7, The groups for the first antigen presentation model (OVA+Vech, OVA+Bio300) received OVA (20 ug OVA (grade: II, Sigma-Aldrich, Saint Louis, MO, USA) emulsified in 2 mg of Aluminum hydroxide in saline solution 100 ul) by Intra-peritoneal injection. The other antigen presentation model included PBS+Veh and PBS+BIO 300™ BIO 300™ groups, which were given PBS). During Day 14 to Day 28, OVA+BIO 300™ BIO 300™ and PBS+BIO 300™ BIO 300™ group received BIO 300™ BIO 300™ (100 ul) by oral gavage at every other day. The other group received control mixture. Moreover, OVA+Veh and OVA+BIO 300™ BIO 300™ received 20 ul of 1% OVA per 7 times via intranasal injection in 14, 16, 18, 20, 22, 24, 26, 28. Another groups received PBS via intranasal. Day 29, airway hyperresponsiveness (AHR) was assessed and tissues were obtained for further analysis.

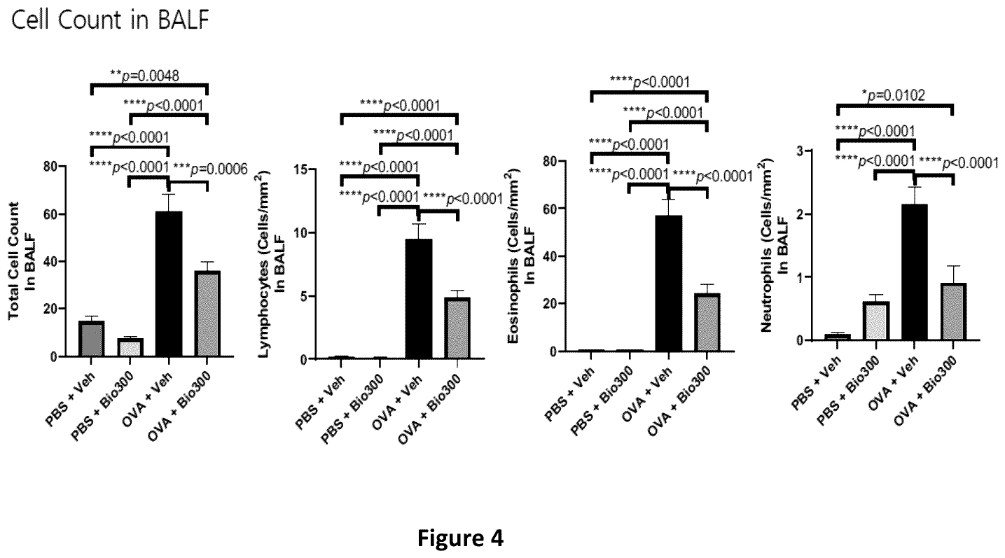

For cell counts in BALF, it was shown that lymphocytes, eosinophils and neutrophils positive cells were significantly decreased when OVA+BIO 300™ was used as compared to OVA+Veh. the eosinophils/lymphocyte results displayed a down ratio in OVA+BIO 300™ as compared to OVA+Veh. Further, the total cell count result showed significantly decreased OVA+BIO 300™ vs OVA+Veh. (FIG. 4). These results indicate that BIO 300™ is capable of decreasing inflammatory cell counts in mice presented with an antigen.

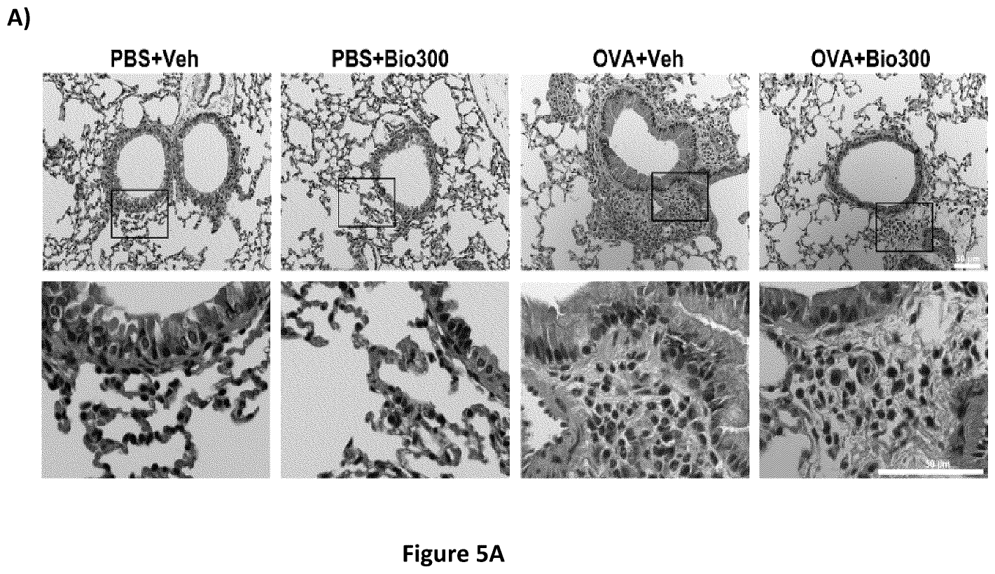

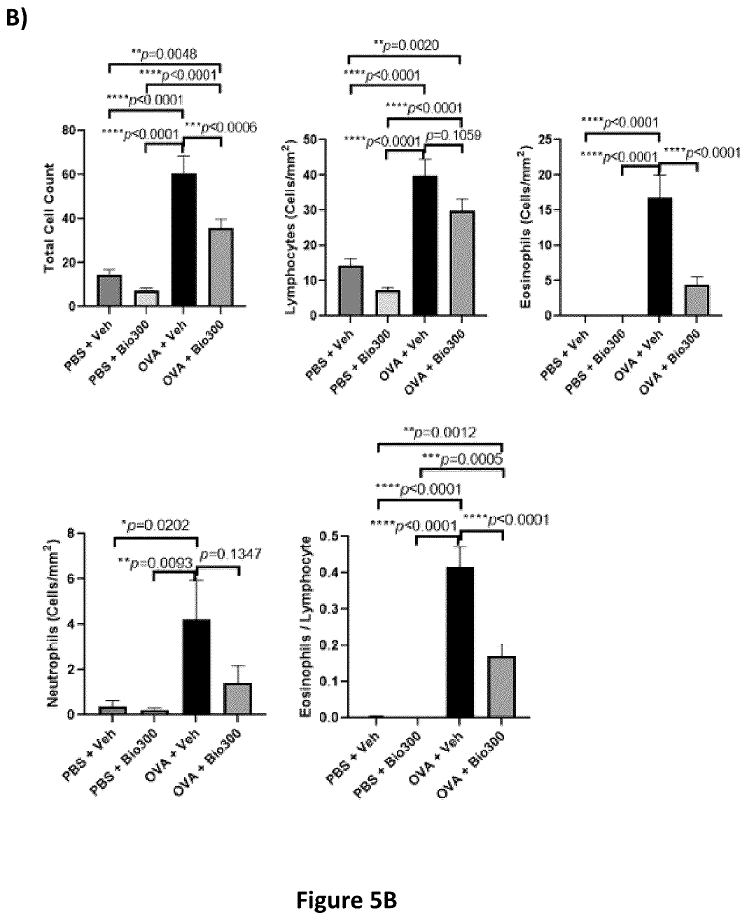

The inventors then examined inflammatory cell counts in tissues. Similarly, the eosinophils and eosinophils/lymphocyte results showed the cell counts were significantly decreased in OVA+BIO 300™ as compared to OVA+Veh. Lymphocytes and Neutrophils positive cells also showed a decreased pattern in OVA+BIO 300™ vs OVA+Veh. Total cell count result showed significantly decreased OVA+BIO 300™ vs OVA+Veh. (FIG. 5A-B). These results further support BIO 300™ being capable of decreasing inflammatory cell counts.

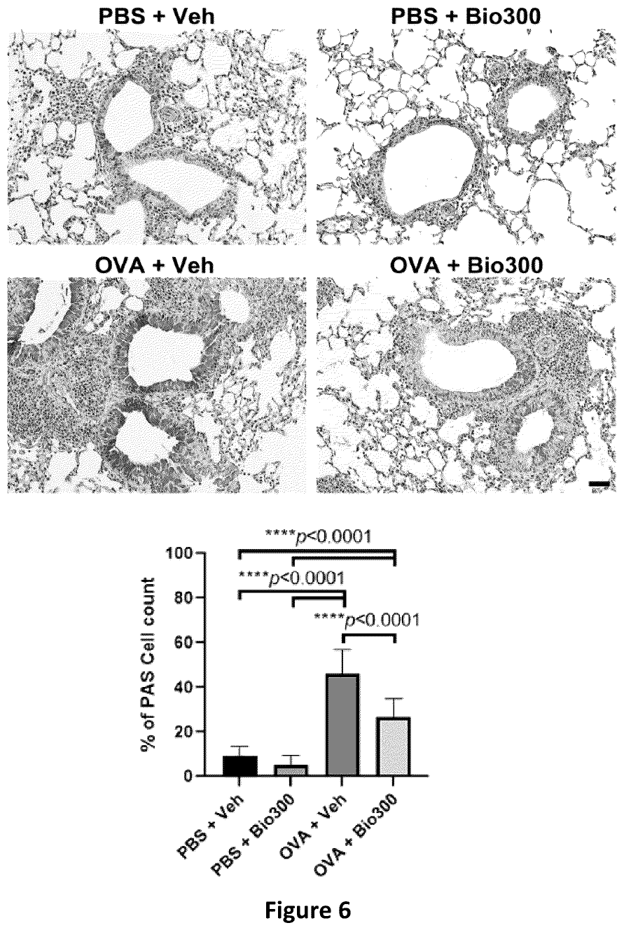

The lung tissue was stained with Periodic Acid-Schiff (PAS) stain to examine goblet cell number. Compared with the control group, OVA+Veh groups showed increased goblet cells. Furthermore, goblet cell number was significantly decreased OVA+BIO 300™ versus OVA+Veh. (FIG. 6) The presence of increased goblet cells suggests glandular remodeling of the airway which leads to increased mucus production, one of the typical findings in severe asthma. The BIO 300™ treated cells showed decreased stimulus for mucus producing cells as they are blocked by BIO 300™.

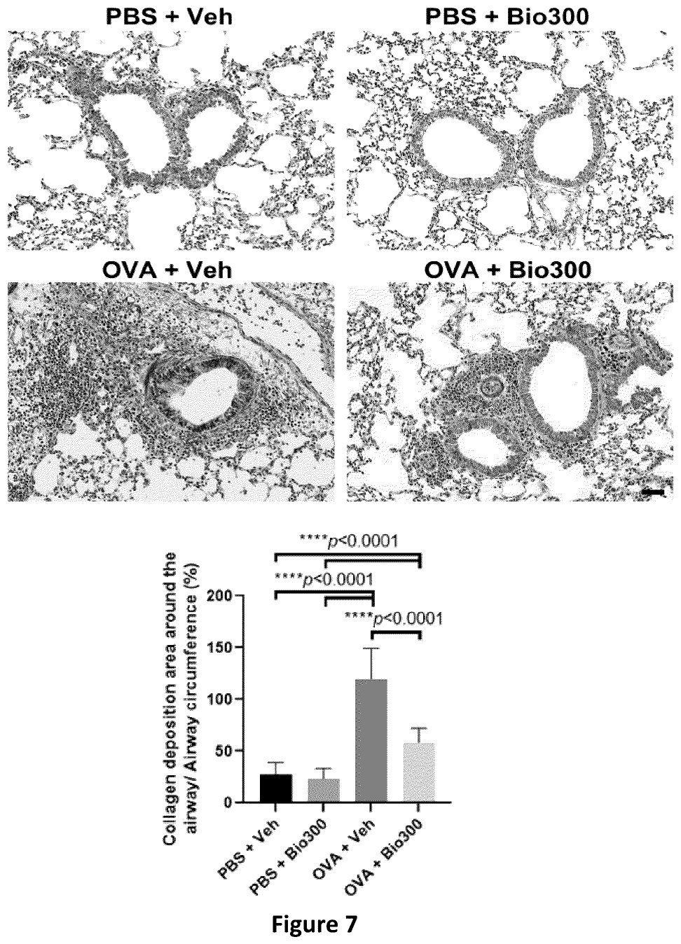

The lung tissue was also stained with Trichrome stain. Compared with the control group, OVA+Veh groups showed increased goblet cells. Furthermore, goblet cell number was significantly decreased in OVA+BIO 300™ as compared to OVA+Veh. (FIG. 7) The increased goblet cells in those cells not treated with BIO 300™ suggest airway submucosal fibrosis which leads to declined lung function in asthma.

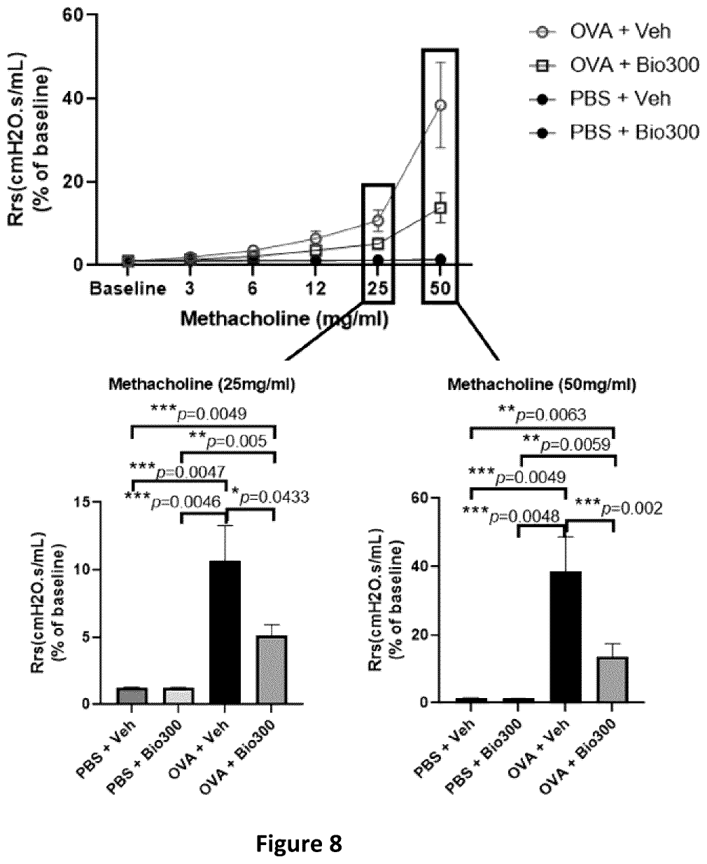

The inventors used total respiratory resistance (Rrs) to assess airway hyperresponsiveness (AHR). In 25 mg/ml methacholine, Rrs percentage was significantly decreased in OVA+BIO 300™ as compared to OVA+Veh. With a 50 mg/ml dose, Rrs percentage was further decreased by about double in OVA+BIO 300™ BIO 300™ as compared to OVA+Veh. (FIG. 8) This physiologic study shows airway hyperactivity and increased airway resistance by methacholine, a unique airway functional changes in asthma. The results imply that there is less airway obstruction in the mouse asthma model when treated with BIO 300™.

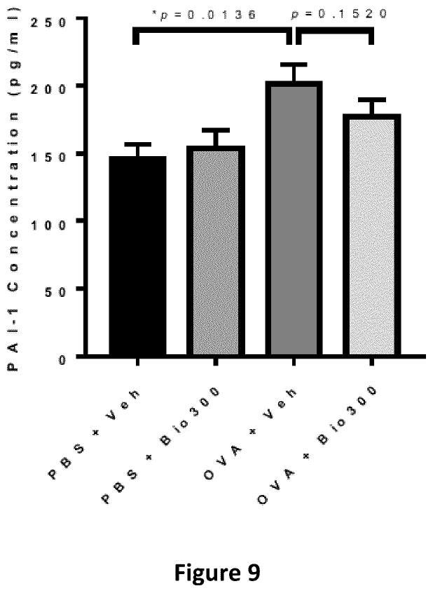

The effect of BIO 300™ treatment of plasminogen activator inhibitor (PAI-1) in BAL fluid (BALF) samples from phosphate-buffered saline (PBS) or OVA-challenged mice was examined. OVA+BIO 300™ BIO 300™ mice were treated with BIO 300™ BIO 300™ during OVA challenge and PAI-1 assessed by ELISA. (FIG. 9). As shown in the graph, administration of BIO 300™ during antigen presentation reduced PAI-1 concentration. In an allergic airway model, less PAI-1 is produced when the animals are treated with BIO 300™ which suggests that BIO 300™ is decreasing the amount PAI-1 response in allergic stimuli.

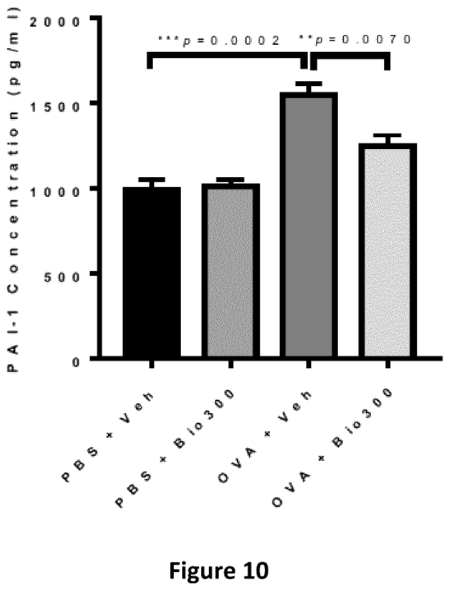

The inventors next examined PAI-1 levels in lung tissue. PAI-1 levels in lung homogenate supernatants were determined by ELISA. PAI-1 concentration was significantly decreased in BIO 300™ treated OVA mice compared to OVA+Veh group. (FIG. 10) This test is a more direct assessment than in the lavage fluid and the results suggest that there is a decrease of PAI-1 levels in the lung tissue.

### Conclusion

In conclusion, BIO 300™ has shown efficacy in treating asthma in a mouse model. BIO 300™ has been shown to decrease inflammatory cells associated with diseases such as asthma. Additionally, BIO 300™ was shown to significantly decrease PAI-1 in both BALF and lung tissue. BIO 300™ may be an efficacious treatment for asthma in adults.

### Example 3-Treatment of Asthma in 4G Genotype Patient with a Genistein Composition (Prophetic)

A 36-year-old male with known asthma on guidelines based therapy presents with poor control associated with shortness of breath, wheezing upon exhaling, and coughing. The subject is diagnosed with poorly controlled asthma. Buccal swabs are taken from the subject and the PAI-1 genotype of the subject is determined. The subject is found to have a 4G4G PAI-1 genotype. The subject is administered a therapeutically effective amount of a composition containing a therapeutically effective amount of genistein in the amount of 32 mg per day for a period of time. The subject's asthma symptoms are alleviated.

### Example 4-Treatment of Asthma in 4G Genotype Patient with BIO 300™ (Prophetic)

A 42-year-old female with known asthma on guidelines based therapy presents with poor control associated with shortness of breath, wheezing upon exhaling, and coughing. The subject is diagnosed with poorly controlled asthma. Buccal swabs are taken from the subject and transcriptomic expression is performed to determine the PAI-1 genotype of the subject. The subject is found to have the 4G5G genotype. The subject is administered a therapeutically effective amount of a BIO 300™ composition containing a therapeutically effective amount of nanoparticulate genistein in the amount of 500 mg per day for a period of time. The subject's asthma symptoms are alleviated.

### Example 5-Treatment of Asthma in Patient with BIO 300™ (Prophetic)

A 34-year-old female with known asthma on guidelines based therapy presents with poor control associated with shortness of breath, wheezing upon exhaling, and coughing. The subject is diagnosed with poorly controlled asthma. The patient is administered as therapeutically effective amount of a BIO 300™ composition containing a therapeutically effective amount of nanoparticulate genistein in the amount of 1500 mg per day for a period of time. The subject's asthma symptoms are alleviated.

### REFERENCES

1. Cesari M, Pahor M, Incalzi R A. Plasminogen activator inhibitor-1 (PAI-1): a key factor linking fibrinolysis and age-related subclinical and clinical conditions. Cardiovasc Ther 2010; 28: e72-91.
2. Zuo Y, Warnock M, Harbaugh A, Yalavarthi S, Gockman K, Zuo M, et al.

Plasma tissue plasminogen activator and plasminogen activator inhibitor-1 in hospitalized COVID-19 patients. Sci Rep 2021; 11:1580.
3. Oh C K, Ariue B, Alban R F, Shaw B, Cho S H. PAI-1 promotes extracellular matrix deposition in the airways of a murine asthma model. Biochem Biophys Res Commun 2002; 294:1155-60.
4. Cho S H, Tam S W, Demissie-Sanders S, Filler S A, Oh C K. Production of plasminogen activator inhibitor-1 by human mast cells and its possible role in asthma. J Immunol 2000; 165:3154-61.
5. Cho S H, Lee S H, Kato A, Takabayashi T, Kulka M, Shin S C, et al. Cross-talk between human mast cells and bronchial epithelial cells in plasminogen activator inhibitor-1 production via transforming growth factor-β1. Am J Respir Cell Mol Biol 2015; 52:88-95.
6. Lee S H, Eren M, Vaughan D E, Schleimer R P, Cho S H. A plasminogen activator inhibitor-1 inhibitor reduces airway remodeling in a murine model of chronic asthma. Am J Respir Cell Mol Biol 2012; 46:842-6.
7. Miyamoto S, Hattori N, Senoo T, Onari Y, Iwamoto H, Kanehara M, et al.

Intra-airway administration of small interfering RNA targeting plasminogen activator inhibitor-1 attenuates allergic asthma in mice. Am J Physiol Lung Cell Mol Physiol 2011; 301: L908-16.
8. Cho S H, Jo A, Casale T, Jeong S J, Hong S J, Cho J K, et al. Soy isoflavones reduce asthma exacerbation in asthmatic patients with high PAI-1-producing genotypes. J Allergy Clin Immunol 2019; 144:109-17.e4.
9. Smith L J, Kalhan R, Wise R A, Sugar E A, Lima J J, Irvin C G, et al. Effect of a soy isoflavone supplement on lung function and clinical outcomes in patients with poorly controlled asthma: a randomized clinical trial. Jama 2015; 313:2033-43.
10. Schoettler N, Strek M E. Recent Advances in Severe Asthma: From Phenotypes to Personalized Medicine. Chest 2020; 157:516-28.
11. Tsantes A E, Nikolopoulos G K, Bagos P G, Bonovas S, Kopterides P, Vaiopoulos G. The effect of the plasminogen activator inhibitor-1 4G/5G polymorphism on the thrombotic risk. Thromb Res 2008; 122:736-42.
12. Wang M, Herbst R S, Boshoff C. Toward personalized treatment approaches for non-small-cell lung cancer. Nat Med 2021; 27:1345-56.
13. Kaur R, Chupp G. Phenotypes and endotypes of adult asthma: Moving toward precision medicine. J Allergy Clin Immunol 2019; 144:1-12.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a method of treating high PAI-1 diseases such as asthma, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A method of decreasing plasminogen activator inhibitor 1 (PAI-1) levels in a patient having a disease characterized by increased plasminogen activator inhibitor 1 (PAI-1) as compared to a normal control comprising:
administering a therapeutically effective amount of a composition comprising nanoparticle genistein and at least one pharmaceutically acceptable carrier to the patient.

2. The method of claim 1, further comprising performing or having performed genotype analysis of the PAI-1 gene in the patient prior to administration of the composition.

3. The method of claim 2, wherein the composition is administered only if the patient is determined to have a 4G allele of the PAI-1 gene.

4. The method of claim 1, wherein the composition is BIO 300™.

5. The method of claim 4, wherein the therapeutically effective amount is between about 500 mg/day to about 1500 mg/day.

6. The method of claim 1, wherein the disease is asthma.

7. A method of inhibiting PAI-1 promotor activity in a patient having a disease characterized by increased plasminogen activator inhibitor 1 (PAI-1) as compared to a normal control comprising:
performing or having performed genotype analysis of the plasminogen activator inhibitor 1 (PAI-1) gene;
determining or having determined presence of a 4G/5G polymorphism in the PAI-1 gene promoter region; and
administering a therapeutically effective amount of a composition comprising genistein to the patient if the 4G/5G polymorphism is present.

8. The method of claim 7, wherein the composition is further comprised of at least one pharmaceutically acceptable carrier.

9. The method of claim 8, wherein the therapeutically effective amount of genistein is at least 32 mg.

10. The method of claim 8, wherein the disease is selected from the group consisting of asthma, atherosclerosis, thrombosis, metabolic syndrome, lung cancer, obesity, type 2 diabetes, hypertension, atopic dermatitis, chronic sinusitis, eosinophilic esophagitis, idiopathic pulmonary fibrosis, lung injury associated fibrosis, severe acute respiratory syndrome coronavirus 2 (SARS-COV-2 or COVID-19), and cardiovascular disease.

11. The method of claim 10, wherein the disease is asthma.

12. A method of treating a disease characterized by increased plasminogen activator inhibitor 1 (PAI-1) levels in a patient in need thereof comprising:
performing or having performed genotype analysis of the plasminogen activator inhibitor 1 (PAI-1) gene; and
administering a therapeutically effective amount of a composition comprising genistein to the patient if the patient has a 4G allele.

13. The method of claim 12, wherein the composition further comprises at least one pharmaceutically acceptable carrier.

14. The method of claim 13, wherein the therapeutically effective amount of genistein is at least 32 mg.

15. The method of claim 12, wherein the composition is comprised of nanoparticle genistein and at least one pharmaceutically acceptable carrier.

16. The method of claim 15, wherein the composition is BIO 300™.

17. The method of claim 16, wherein the therapeutically effective amount is between about 500 mg/day to about 1500 mg/day.

18. The method of claim 14, wherein the disease is selected from the group consisting of asthma, atherosclerosis, thrombosis, metabolic syndrome, lung cancer, obesity, type 2 diabetes, hypertension, atopic dermatitis, chronic sinusitis, eosinophilic esophagitis, idiopathic pulmonary fibrosis, lung injury associated fibrosis, severe acute respiratory syndrome coronavirus 2 (SARS-COV-2 or COVID-19), and cardiovascular disease.

19. The method of claim 18, wherein the disease is asthma.
